(19) United States
(12) Patent Application Publication
(10) Pub. No.: US 2025/0093258 A1
(43) Pub. Date: Mar. 20, 2025

(54) DIAGNOSTIC PLATFORM FOR ANALYZING AND OPTIMIZING WELL TREATMENT FLUIDS

(71) Applicant: Board of Regents, The University of Texas System, Austin, TX (US)

(72) Inventors: Carlos Torres-Verdín, Austin, TX (US); Ayaz Mehmani, Austin, TX (US); Colin Schroeder, Austin, TX (US)

(73) Assignee: Board of Regents, The University of Texas System, Austin, TX (US)

(21) Appl. No.: 18/291,539

(22) PCT Filed: Jul. 18, 2022
(86) PCT No.: PCT/US2022/073851

§ 371 Date: Jan. 23, 2024

Related U.S. Application Data
(60) Provisional application No. 63/224,932, filed on Jul. 23, 2021.

(51) Int. Cl. G01N 21/05; B01L 3/00; G01N 21/03; G01N 21/85; G01N 33/28

(52) U.S. Cl. CPC G01N 21/05; B01L 3/50273; G01N 21/0332; G01N 21/85; G01N 33/2823; B01L 2200/027; B01L 2300/0663; B01L 2300/0864; B01L 2300/1805; B01L 2400/0487

(57) ABSTRACT

Systems, methods, and techniques are described herein for characterizing well treatment fluids and well treatment fluid performance. For example, well treatment fluids can be characterized using microfluidic chips to determine fluid properties, with very small amounts of fluid used for the characterization process. Optical analysis and electrical conductivity analysis of well treatment fluids as they pass through different microfluidic pathways with different cross-sectional dimensions can be used to gain information about the well treatment fluid's composition, structure, and performance. In some cases, microfluidic pathways can be functionalized as a model for a wellbore and a reservoir (e.g., rock in the reservoir) to evaluate the performance of well treatment fluids by imaging the well treatment fluid as it flows into or through the functionalized model. These systems, methods, and techniques can allow for optimization of well treatment fluids prior to or during drilling or completion operations.

### CROSS-REFERENCE TO RELATED APPLICATIONS

This application claims the benefit of and priority to U.S. Provisional Application No. 63/224,932, filed on Jul. 23, 2021, which is hereby incorporated by reference in its entirety.

### FIELD

This invention is in the field of well treatment fluid analysis and optimization. This invention relates generally to systems, methods, and techniques for on-site analysis and optimization of well treatment fluids, such as drilling mud, cleaning fluids, casing fluids, etc., using microfluidic chips.

### BACKGROUND

Well drilling operations can introduce contamination into an oil-bearing rock formation. For example, porous and permeable formations are susceptible to mud-filtrate invasion during overbalanced drilling when the pressure maintained in the borehole by dense drilling mud exceeds the formation pressure. As high-pressure drilling mud is pressed against a porous and permeable formation, solid particles from the drilling mud are filtered out and accumulate at the borehole wall forming an external mudcake. Smaller particles in the drilling fluid may also migrate into the pore space around the wellbore forming an internal mudcake. Simultaneously, the liquid component of the drilling mud, known as mud filtrate, can flow into the formation and displace or mix with the in situ reservoir fluids. Thus, petrophysical properties and fluid saturations in the near-wellbore region may be altered, causing borehole measurements acquired shortly after drilling to represent properties of the invaded zone rather than the undisturbed reservoir. Further, deposition of internal mudcake around the wellbore can irreversibly damage the hydrocarbon-bearing formation, potentially hindering both short- and long-term well productivity. To limit these potentially detrimental effects and to ensure that wells are drilled economically and safely, drilling and treatment fluid compositions can be continuously monitored and optimized as drilling progresses.

An ideal drilling mud is able to keep the drill bit cool, prevent the entrance of formation fluids into the wellbore, limit the loss of mud-filtrate to the formation, remove cuttings from the well, suspend cuttings when drilling is paused, and maintain consistent physical properties when exposed to contamination. Mud composition, however, does not remain constant while drilling a wellbore. A change in mud composition may occur due to mixing of drilling mud with formation fluids and formation particles such as clays, elevated shear stress as the mud circulates through the drill string and drill bit, changes in temperature and pressure, and loss of mud filtrate into the formation. Current mud analysis methods are outdated, inconsistent, expensive, and time-consuming to perform. Further, these measurements require space that may not be available when drilling offshore or in harsh environments and are generally unable to measure the mud properties at in situ conditions. Thus, given that drilling mud is a complex fluid, it is very challenging to predict its behavior in the borehole using traditional measurement equipment. Mud engineers therefore may not have the necessary information to adjust the mud composition promptly and appropriately.

### SUMMARY

Described herein are methods and systems relating to point-of-care diagnostic tools and useful for monitoring and optimizing the composition of and understanding performance properties of well treatment fluids, such as drilling mud. The disclosed methods and systems can be used either prior to or during drilling operations and may use only a minimal amount of fluid volume (e.g., less than 5 ml), and can return results within minutes, with limited operator training and supervision needed.

Well treatment fluid property measurement tools can be costly and require space such that they are difficult to operate in harsh drilling environments. The methods and systems described herein provide a multifaceted platform that can decompose the well treatment fluid into classes based on its constituent particle sizes. Each class may comprise particles with specific physical and chemical properties that can be representative of a timeline for the well treatment fluid composition as it, along with its particle sizes, changes during drilling or completion operations. In other words, by separating the well treatment fluid into particle size-based classes, fluid properties such as rheometry, emulsion quality, and clay detachment effects can be captured and predicted as composition is altered during drilling (with smaller channels representing future steps). The rate of fluid composition change can be determined by taking samples at low frequency intervals. The systems and methods described herein are useful for characterizing fluid properties and do not require a cleanroom and are compatible with harsh and dusty environments, such as may be present at a drilling rig or site.

Methods and systems described herein are also useful for diagnosing how well treatment fluids may behave when used in a drilling or completion operation, such as to determine fluid loss metrics, mudcake formation potential, and formation damage potential. The disclosed methods and systems can replace traditional drilling fluid loss tests performed at the rig site, such as traditional tests involving filtering a pressurized mud sample through filter paper for a period of 30 minutes and then measuring the thickness of the resulting mudcake and the volume of mud-filtrate collected. Although results from such tests can be used to estimate the rate that permeable formations are sealed by mudcake after being penetrated by the drill bit, they fail to account for the influence of formation properties and therefore may bear little resemblance to actual conditions occurring in the borehole and surrounding reservoir. The disclosed methods and systems provide for analyzing treatment fluids designed to reverse the effects of formation damage caused during drilling and allow analysis to be performed easily and quickly on site.

The disclosed methods and systems employ a microfluidics-based platform for optimizing drilling mud and treatment fluid composition by evaluating performance characteristics using microfluidic chips functionalized as micromodels for a wellbore and formation. The micromodels can comprise a porous system that resembles the formation. Micromodel fabrication can be completed remotely and micromodels can be shipped to the operator onsite, such as in the form of a library of micromodels corresponding to a variety of pore structures, allowing an appropriate micromodel to be selected, such as using information gathered from geological interpretations, stratigraphy, core samples from nearby wells, and cuttings.

In a first aspect, methods and systems for characterizing well treatment fluids are disclosed herein. In an example, a method of this aspect may comprise providing a microfluidic chip, such as a microfluidic chip comprising at least one inlet, a plurality of outlets, and a channel array comprising plurality of microfluidic channels having different cross-sectional dimensions, such as where the at least one inlet is in fluid communication with the plurality of microfluidic channels, and where the plurality of microfluidic channels are in respective fluid communication with the plurality of outlets. In some examples, one or more electrical contact points or electrodes may be included in the microfluidic channels to allow for sensing of electrical conductivity of the well treatment fluid in the microfluidic channels. In some examples, multiple electrical contact points or electrodes may be included at various points along the length of a microfluidic channel, which can be useful for monitoring electrical conductivity of the well treatment fluid, which may give insights as to compositional changes of the well treatment fluid as it flows through the microfluidic channel. In some examples, a microfluidic chip and/or microfluidic channels in a microfluidic chip may include an array of electrical contact points. Methods of this aspect may comprise or further comprise flowing a well treatment fluid into the at least one inlet to pass the well treatment fluid to the plurality of microfluidic channels. For example, the well treatment fluid may partition into a plurality of different size classes based on a cross-sectional dimension of a respective microfluidic channel. Methods of this aspect may comprise or further comprise obtaining imaging data and/or electrical conductivity data of flows of the well treatment fluid in the plurality of microfluidic channels and characterizing fluid properties for the well treatment fluid or for each of the different size classes using the imaging data. Imaging devices such as microscopes or cameras may be useful for obtaining the imaging data. In some examples, obtaining the imaging data of flows of the well treatment fluid in the plurality of microfluidic channels comprises imaging positions of the well treatment fluid in the plurality of microfluidic channels as a function of time.

Optionally, a pressure pump providing an adjustable pressure and flow rate can be used to control the transport and/or pressure of the well treatment fluid into the plurality of microfluidic channels. In some cases, the pressure and/or flow rate provided by the pressure pump can be controlled in real-time. In some cases, controlling temperature and/or pressure of the microfluidic channels may be desirable. In some examples, methods of this aspect may comprise or further comprise controlling a temperature of the plurality of microfluidic channels to obtain temperature dependent imaging and/or electrical conductivity data of flows of the well treatment fluid in the plurality of microfluidic channels. Optionally, characterizing the fluid properties comprises using the temperature dependent imaging and/or electrical conductivity data. In some examples, methods of this aspect may comprise or further comprise monitoring pressures at the plurality of microfluidic channels while obtaining the imaging and/or electrical conductivity data. Optionally, characterizing the fluid properties comprises using the imaging and/or electrical conductivity data and the pressures. In some examples, methods of this aspect may comprise or further comprise controlling a backpressure at the plurality of outlets to obtain pressure dependent imaging and/or electrical conductivity data of flows of the well treatment fluid in the plurality of microfluidic channels. Optionally, characterizing the fluid properties comprises using the pressure dependent imaging and/or electrical conductivity data.

Machine learning models are useful with the methods and systems of this aspect. Machine learning models may allow for rapidly transforming obtained imaging and/or electrical conductivity data to fluid properties, such as by first training the machine learning model using reference fluids with known fluid properties and corresponding imaging and/or electrical conductivity data used by flowing the reference fluid through a microfluidic chip, as described herein.

In some examples, characterizing the fluid properties comprises applying the imaging and/or electrical conductivity data as input to a trained machine learning model for determining well treatment fluid properties. For example, the trained machine learning model may optionally comprise a set of parameters that were learned using a set of reference fluids, each reference fluid of the set of reference fluid corresponding to a previously characterized well treatment fluid or well treatment fluid component, and parameters of the set of parameters describing fluid properties of one or more reference fluids; and one or more functions configured to transform the input into predicted fluid properties using the set of parameters. In examples, the trained machine learning model generates outputs comprising the fluid properties for the well treatment fluid or for each of the different size classes. Optionally, the input comprises one or more of pressures at the plurality of microfluidic channels, temperature dependent imaging and/or electrical conductivity data, or pressure dependent imaging and/or electrical conductivity data.

Methods and systems of this aspect are useful with a variety of well treatment fluids. In some examples, well treatment fluid comprises drilling mud, a cleaning fluid, a casing fluid, or a reservoir fluid. Optionally, the well treatment fluid may include one or more additives to allow for enhanced characterization and/or to improve the detection schemes described here. Example additives may include optical contrast agents (e.g., ultraviolet absorbing dyes, infrared absorbing dyes, visible absorbing dyes, fluorescent compositions, etc.) or electrical conductivity modifiers (e.g., dissolved salts or organic compounds, buffers, metal nanoparticles, pH modifiers, etc.), which may be useful for enhancing detection using optical or electrical conductivity detection methods. In some examples, one or more optical contrast agents may be introduced to the well treatment fluid as an additive prior to introduction into the microfluidic channels, such as to improve the detectability of the advancement front of the well treatment fluid as it flows through the microfluidic channels over time. Methods and systems of this aspect may be useful for evaluating fluid properties, such as one or more rheological properties, one or more chemical properties, or one or more physical properties. Example fluid properties may comprise one or more of a particle size, a size distribution of particles, an alkalinity, a viscosity, an opacity, or an electrical conductivity.

In some examples, methods of this aspect may comprise or further comprise collecting outflow from one or more of the plurality of outlets and applying a chemical analysis or physical analysis to the outflow. Such chemical or physical analysis may be useful for interpreting or verifying the fluid properties determined by the disclosed methods and systems described herein and may optionally be used in a process of modifying a well treatment fluid.

In another example, a system for characterizing well treatment fluids in accordance with this aspect may comprise a microfluidic chip or a receiver, support, or interface for a microfluidic chip. For example, a microfluidic chip may comprise at least one inlet and a plurality of outlets, and a channel array comprising plurality of microfluidic channels having different cross-sectional dimensions, such as where at least one inlet is in fluid communication with the plurality of microfluidic channels, and where the plurality of microfluidic channels are in respective fluid communication with the plurality of outlets. In some examples, one or more electrical contact points or electrodes may be included in the microfluidic channels to allow for sensing of electrical conductivity of the well treatment fluid in the microfluidic channels. In some examples, multiple electrical contact points or electrodes may be included at various points along the length of a microfluidic channel, which can be useful for monitoring electrical conductivity of the well treatment fluid, which may give insights as to compositional changes of the well treatment fluid as it flows through the microfluidic channel. In some examples, a microfluidic chip and/or microfluidic channels in a microfluidic chip may include an array of electrical contact points. In some examples, systems of this aspect may comprise or further comprise a pump in fluid communication with at least one inlet of the microfluidic chip or the receiver, support, or interface of the microfluidic chip for passing the well treatment fluid to the plurality of microfluidic channels to partition the well treatment fluid into a plurality of different size classes based on a cross-sectional dimension of a respective microfluidic channel; an imaging sensor in optical communication with the microfluidic chip or the receiver, support, or interface of the microfluidic chip, for obtaining imaging data of flows of the well treatment fluid in the plurality of microfluidic channels; and a computing system for analyzing the imaging and/or conductivity data to characterize fluid properties for the well treatment fluid or for each of the different size classes. The computing system may be optionally be in electrical communication with one or more electrodes or electrical contact points in the microfluidic chip or microfluidic channels therein. Optionally the computing system may be used to directly sense electrical conductivity data.

Systems of this aspect may optionally comprise one or more sensors or controllers for monitoring or controlling various physical properties associated with the microfluidic chip or the microfluidic channels. Optionally, a pressure pump providing an adjustable pressure and flow rate can be used to control the pressure and/or transport of the well treatment fluid into the microfluidic chip or the microfluidic channels. In some cases, the pressure and/or flow rate provided by the pressure pump can be controlled in real-time. In some examples, systems of this aspect may comprise or further comprise one or more of: a pressure transducer coupled to the microfluidic chip for determining pressures in the plurality of microfluidic channels; a heater in thermal communication with the microfluidic chip for controlling a temperature in the plurality of microfluidic channels; or a light source in optical communication with the microfluidic chip for illuminating the plurality of microfluidic channels.

Computing systems of systems described herein may comprise a processor in data communication with the imaging sensor, and a non-transitory computer readable storage medium in data communication with the processor, the non-transitory computer readable storage medium comprising instructions that, when executed by the processor, cause the processor to perform operations. Example operations may be associated with the methods described herein. In some specific examples, the operations may comprise obtaining imaging data of flows of the well treatment fluid in the plurality of microfluidic channels using the imaging sensor; and characterizing fluid properties for the well treatment fluid or for each of the different size classes using the imaging data. In some examples, the operations may include or further include obtaining electrical conductivity data of the well treatment fluid in the plurality of microfluidic channels. In some examples, the operations may include or further include controlling a temperature of the plurality of microfluidic channels using a heater to obtain temperature dependent imaging and/or electrical conductivity data of flows of the well treatment fluid in the plurality of microfluidic channels. Optionally, characterizing the fluid properties comprises using the temperature dependent imaging and/or electrical conductivity data. In some examples, the operations may include or further include monitoring pressures at the plurality of microfluidic channels using a pressure transducer while obtaining the imaging and/or electrical conductivity data. Optionally, characterizing the fluid properties comprises using the imaging and/or electrical conductivity data and the pressures.

Machine learning models may be useful with the systems described herein. In some examples, characterizing the fluid properties comprises applying the imaging data as input to a trained machine learning model for determining well treatment fluid properties. As described above, the trained machine learning model may comprise a set of parameters that were learned using a set of reference fluids, each reference fluid of the set of reference fluid corresponding to a previously characterized well treatment fluid or well treatment fluid component, and parameters of the set of parameters describing fluid properties of one or more reference fluids; and one or more functions configured to transform the input into predicted fluid properties using the set of parameters. In examples, the trained machine learning model generates outputs comprising the fluid properties for the well treatment fluid or for each of the different size classes. Optionally, the input comprises one or more of imaging and/or electrical conductivity data, pressures at the plurality of microfluidic channels, temperature dependent imaging and/or electrical conductivity data, or pressure dependent imaging and/or electrical conductivity data.

In another aspect, methods and systems for characterizing performance of well treatment fluids are disclosed herein. In an example, a method of this aspect may comprise providing a microfluidic chip, such as a microfluidic chip comprising an inlet and an outlet, and a microfluidic channel in fluid communication with and between the inlet and the outlet and functionalized as a model for a wellbore and a reservoir; flowing a well treatment fluid into the inlet to pass the well treatment fluid to the microfluidic channel; obtaining imaging and/or electrical conductivity data of flow of the well treatment fluid in the microfluidic channel; and characterizing performance properties of the well treatment fluid in the microfluidic channel using the imaging and/or electrical conductivity data. In examples, the performance properties of the well treatment fluid in the microfluidic channel comprises one or more of characteristics of an external mudcake in a wellbore portion of the microfluidic channel, characteristics of an internal mudcake in a reservoir portion of the microfluidic channel, characteristics of a mud filtrate invasion zone in the reservoir portion of the microfluidic channel, or characteristics of formation damage in the reservoir portion of the microfluidic channel. Optionally, providing the microfluidic chip includes selecting, obtaining, or receiving the microfluidic chip from a microfluidic chip library. In some cases, providing the microfluidic chip includes fabricating the microfluidic chip.

As with the methods for determining fluid properties, methods of this aspect may include monitoring or controlling various physical properties, such as pressure or temperature. In some examples, methods of this aspect may comprise or further comprise controlling a temperature of the microfluidic channel to obtain temperature dependent imaging and/or electrical conductivity data of flow of the well treatment fluid in the microfluidic channel. Optionally, characterizing performance properties of the well treatment fluid comprises using the temperature dependent imaging and/or electrical conductivity data. In some examples, methods of this aspect may comprise or further comprise monitoring pressure at the microfluidic channel while obtaining the imaging and/or electrical conductivity data. Optionally, characterizing performance properties of the well treatment fluid comprises using the imaging and/or electrical conductivity data and the pressure. In some examples, methods of this aspect may comprise or further comprise controlling a backpressure at the outlet to obtain pressure dependent imaging and/or electrical conductivity data of flows of the well treatment fluid in the microfluidic channel. Optionally, characterizing performance properties of the well treatment fluid comprises using the pressure dependent imaging and/or electrical conductivity data.

Machine learning models are useful with the methods and systems of this aspect. Machine learning models may allow for rapidly transforming obtained imaging data to fluid performance properties, such as by first training the machine learning model using reference fluids with known fluid performance properties and corresponding imaging and/or electrical conductivity data used by flowing the reference fluid through a microfluidic chip, as described herein. In some examples, characterizing performance properties of the well treatment fluid comprises applying the imaging and/or electrical conductivity data as input to a trained machine learning model for determining well treatment fluid performance properties. In some examples, the trained machine learning model comprises a set of parameters that were learned using a set of reference fluids, each reference fluid of the set of reference fluid corresponding to a previously characterized well treatment fluid or well treatment fluid component, and parameters of the set of parameters describing performance properties of the one or more reference fluids; and one or more functions configured to transform the input into expected performance properties. Optionally, the input comprises one or more of: imaging and/or electrical conductivity data, pressure at the microfluidic channel, temperature dependent imaging and/or electrical conductivity data, pressure dependent imaging and/or electrical conductivity data, well treatment fluid properties, or composition for the well treatment fluid.

In some cases, machine learning models may be trained to identify desired compositional changes or alternative well treatment fluid compositions and may generate outputs identifying additives for adding to the well treatment fluid, such as to improve performance of the well treatment fluid.

In another example, a system for characterizing well treatment fluid performance in accordance with this aspect may comprise a microfluidic chip or a receiver, support, or interface for a microfluidic chip. An example microfluidic chip may comprise an inlet and an outlet, and a microfluidic channel in fluid communication with and between the inlet and the outlet, such as a microfluidic channel that is functionalized as a model for a wellbore and a reservoir; a pump in fluid communication with the inlet for flowing a well treatment fluid to the microfluidic channel; an imaging sensor in optical communication with the microfluidic chip for obtaining imaging data of flow of the well treatment fluid in the microfluidic channel. In some examples, one or more electrical contact points or electrodes may be included in the microfluidic channels to allow for sensing of electrical conductivity of the well treatment fluid in the microfluidic channels. In some examples, multiple electrical contact points or electrodes may be included at various points along the length of a microfluidic channel, which can be useful for monitoring electrical conductivity of the well treatment fluid, which may give insights as to compositional changes of the well treatment fluid as it flows through the microfluidic channel. In some examples, a microfluidic chip and/or microfluidic channels in a microfluidic chip may include an array of electrical contact points. Systems of this aspect may further comprise a computing system for analyzing the imaging and/or electrical conductivity data to characterize performance properties of the well treatment fluid.

Systems of this aspect may optionally comprise one or more sensors or controllers for monitoring or controlling various physical properties associated with the microfluidic chip or the microfluidic channels. In some examples, systems of this aspect may comprise or further comprise one or more of a pressure transducer coupled to the microfluidic chip for determining pressure in the microfluidic channel; a heater in thermal communication with the microfluidic chip for controlling a temperature in the microfluidic channel; or a light source in optical communication with the microfluidic chip for illuminating the microfluidic channel.

Computing systems of systems described herein may comprise a processor in data communication with the imaging sensor, and a non-transitory computer readable storage medium in data communication with the processor, the non-transitory computer readable storage medium comprising instructions that, when executed by the processor, cause the processor to perform operations. Example operations may be associated with the methods described herein. In some specific examples, the operations may comprise obtaining imaging and/or electrical conductivity data of flow of the well treatment fluid in the microfluidic channels using the imaging sensor or electrodes or contact points; and characterizing performance properties of the well treatment fluid using the imaging and/or electrical conductivity data. In some examples, the operations may include or further include controlling a temperature of the microfluidic channel using a heater to obtain temperature dependent imaging and/or electrical conductivity data of flows of the well treatment fluid in the microfluidic channel. Optionally, characterizing performance properties of the well treatment fluid comprises using the temperature dependent imaging and/or electrical conductivity data. In some examples, the operations may include or further include monitoring pressure at the microfluidic channel using a pressure transducer while obtaining the imaging and/or electrical conductivity data. Optionally, characterizing performance properties of the well treatment fluid comprises using the imaging and/or electrical conductivity data and the pressures.

Machine learning models may be useful with the systems described herein. In some examples, characterizing performance properties of the well treatment fluid comprises applying the imaging and/or electrical conductivity data as input to a trained machine learning model for determining fluid performance properties. Optionally, a trained machine learning model comprises a set of parameters that were learned using a set of reference fluids, each reference fluid of the set of reference fluid corresponding to a previously characterized well treatment fluid or well treatment fluid component, and parameters of the set of parameters describing performance properties of one or more reference fluids; and one or more functions configured to transform the input into a predicted well treatment fluid performance using the set of parameters. In some examples, the input may comprise one or more of and/imaging or electrical conductivity data, pressure at the microfluidic channel, temperature dependent imaging and/or electrical conductivity data, pressure dependent imaging and/or electrical conductivity data, well treatment fluid properties, or composition for the well treatment fluid.

It will be appreciated that the methods and systems for characterizing well treatment fluids are usable and combinable with the methods and systems for characterizing performance of well treatment fluids. Such combination can advantageously allow for a more robust process of optimizing well treatment fluids by iteratively characterizing them and characterizing their performance, allowing changes to the well treatment fluid to be made, observing the fluid properties and fluid performance properties prior to using the well treatment fluid in a drilling or completion operation, for example.

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 provides a schematic illustration of an example system for characterizing and evaluating performance of well treatment fluids.

FIG. 2 provides a schematic illustration of an example section of a microfluidic chip for characterizing well treatment fluids.

FIG. 3 provides an overview of an example method for characterizing well treatment fluids.

FIG. 4 provides a schematic illustration of an example section of a microfluidic chip functionalized as a model for a wellbore and a formation for evaluating well treatment fluid performance.

FIG. 5 provides an overview of an example method for characterizing well treatment fluid performance.

### DETAILED DESCRIPTION

Various systems, methods, and techniques are described herein for characterizing well treatment fluids and well treatment fluid performance. For example, well treatment fluids can be characterized using microfluidic chips to determine fluid properties, with very small amounts of fluid used for the characterization process. Optical analysis of well treatment fluids as they pass through different microfluidic pathways with different cross-sectional dimensions can be used to gain information about the well treatment fluid's composition, structure, and performance. In some cases, microfluidic pathways can be functionalized as a model for a wellbore and a reservoir (e.g., rock in the reservoir) to evaluate the performance of well treatment fluids. These systems, methods, and techniques can allow for optimization of well treatment fluids prior to or during drilling or completion operations.

Various well treatment fluids can be analyzed using the systems, methods, and techniques disclosed herein. Without limitation, the well treatment fluid may comprise drilling mud, a cleaning fluid, a casing fluid, or a reservoir fluid. It will be appreciated that each of these fluids may comprise a plurality of different components or additives, including solid particles, aqueous mixtures, non-aqueous mixtures, dispersions, suspensions, emulsions, continuous phases, dispersed phases, or the like.

In general the terms and phrases used herein have their art-recognized meaning, which can be found by reference to standard texts, journal references and contexts known to those skilled in the art. The following definitions are provided to clarify their specific use in the context of the invention.

“Fluid bearing formation” refers to any subterranean rock formation that contains liquid or gaseous fluids or mixtures of liquid and gaseous fluids (also referred to as a “formation” or a “reservoir”). A specific example of a fluid bearing formation is an oil bearing formation, which may contain liquid and/or gaseous hydrocarbons. A formation may include any type of mineral structure or composition associated with petroleum production from land-based wells and/or undersea wells, for example.

“External mudcake” refers to deposited components of a drilling mud on the walls of a wellbore that serve to isolate a fluid bearing formation from drilling fluids in the wellbore. An “internal mudcake” refers to those deposited components of a drilling mud that penetrate into the fluid bearing formation adjacent to the wellbore. “Filtrate” refers to liquid components of the drilling mud that penetrate into the fluid bearing formation.

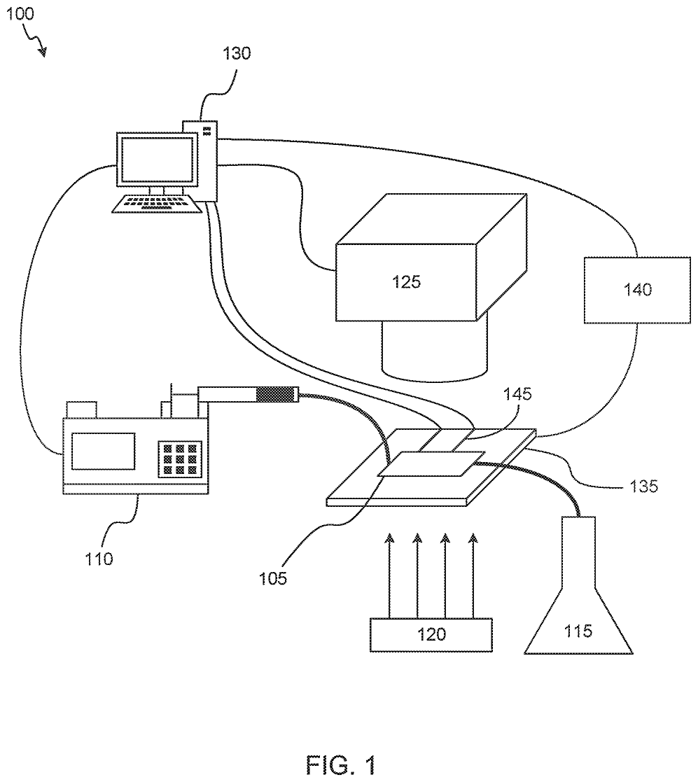

FIG. 1 provides a schematic illustration of an example system 100 for characterizing and evaluating performance of well treatment fluids. System 100 includes a microfluidic chip 105 in fluid communication with a pump 110 and an effluent collection vessel 115. The pump 110 may comprise a syringe pump, pressure pump, or other pumping device for providing a well treatment fluid to one or more inlets of microfluidic chip 105. Pump 110 may allow for control over the well treatment fluid pressure and/or the flow rate of the well treatment fluid into or through the microfluidic chip 105 in real-time. Effluent collection vessel 115 may comprise one or more vessels for collecting outflow from one or more outlets of the microfluidic chip 105, allowing for later chemical or physical analysis of the outflow, for example.

System 100 also includes a light source 120 and an imaging sensor 125 in optical communication with microfluidic chip 105. Imaging sensor 125 can be used to obtain imaging data of flows of the well treatment fluid in or across microfluidic chip 105. Light source 120 can be positioned in a trans-illumination configuration to allow light from light source 120 to be directed to imaging sensor 125 through microfluidic chip 105. In some cases, light source 120 can be positioned in an epi-illumination or other configuration to allow light from light source 120 to be scattered or reflected from microfluidic chip 105 or otherwise illuminate microfluidic chip 105 for imaging by imaging sensor 125. In some examples, imaging sensor 125 can be or comprise a camera or microscope system and allow for obtaining imaging data, optionally corresponding to images or pictures of microfluidic chip 105, as a function of time as the well treatment fluid flows in or across microfluidic chip 105. Optionally, one or more optical contrast agents may be introduced to the well treatment fluid as an additive prior to introduction into the microfluidic channels, such as to improve the detectability of the advancement front of the well treatment fluid by the imaging sensor 125 as fluid flows through the microfluidic chip 105 over time.

System 100 also includes a computer system 130, which can comprise, without limitation, a processor, a non-transitory computer readable storage medium, a display device, an input device, an output device, or the like. Computer system 130 is shown in data communication with imaging device 125, such as for receiving the imaging data collected using imaging device 125 and for analysis of the imaging data. Computer system 130 is shown in data communication with pump 110, such as for controlling pumping rate, pressure, or the like of well treatment fluid in microfluidic chip 105.

Computer system 130 may also be in data communication with other sensors or control components. For example, platform 135 may support microfluidic chip 105 and may contain a heater, a heat pump (e.g. a Peltier device), or other components for adding heat to or removing heat from microfluidic chip 105 so as to effect or control temperature of the well treatment fluid and/or microfluidic chip 105. A temperature controller 140 may allow for adjustment and sensing of the temperature using the components of platform 135. In some examples, temperature controller 140 may be a component of or integrated as part of computer system 130. As another example, microfluidic chip 105 may contain or be arranged in fluid communication with one or more pressure transducers (not shown in FIG. 1) in data communication with computer system 130, such as for monitoring one or more pressures within microfluidic chip 105. Microfluidic chip 105 may also include one or more actuatable valves or plugs, such as in control communication with computer system 130, to control flow of well treatment fluid within or across microfluidic chip 105. As another example, electrodes 145 or electrical contact points may be included in microfluidic chip 105, such as to allow electrical contact with well treatment fluid flowing through microfluidic chip 105, and electrodes 145 or electrical contact points may be in electrical communication with computer system 130 to allow for conductivity measurements of the well treatment fluid to be determined.

A microfluidic chip, such as microfluidic chip 105, may be structured to include one or more fluid pathways, such as between one or more inlets and one or more outlets. Well treatment fluid can be introduced into an inlet of a microfluidic chip and monitored as it flows through the fluid pathway(s) using an imaging device or electrical conductivity measurements, as described above. By structuring the microfluidic chip appropriately, information about the well treatment fluid can be discerned based on the imaging data acquired by the imaging device. Additionally, the imaging data can be varied based on a temperature and pressure of the treatment fluid, which can be controlled to allow temperature dependent imaging data and/or pressure dependent imaging data to be acquired. For example, the imaging data can be used to determine fluid properties or evaluate how the well treatment fluid might perform in a formation adjacent to a wellbore.

Microfluidic chips can be prepared using any suitable techniques. For example, the microfluidic chip may be prepared using fabrication techniques including, but not limited to, photolithography, electron beam lithography, masking, etching, atomic or chemical layer deposition, soft lithography, laser ablation, microcontact printing, replica molding, embossing, injection molding, or the like. Microfluidic chips can comprise any suitable material, such as glass, polymer, dielectric, silicon, or the like. In some cases, microstructures in a fluid pathway of a microfluidic chip can be generated to mimic, model, or represent porosity features of rock in a formation, such as pore sizes, pore structures, which can be useful for understanding how different well treatment fluids can flow in different types of rock. In some cases, fluid pathway surfaces may be structured to alter the fluid flow properties or may be coated or treated with various chemical agents or functionalized to allow for interaction with various fluid components or modification of various fluid components so as to further investigate different chemical or physical properties of a well treatment fluid.

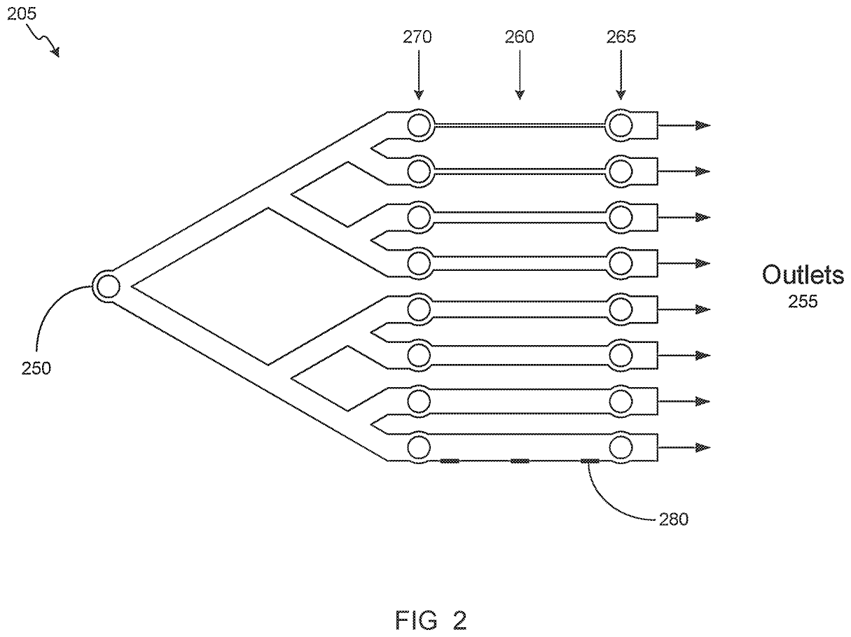

FIG. 2 provides a schematic illustration of an example section of a microfluidic chip 205, useful for characterizing well treatment fluids in association with the methods, systems, and techniques described herein. Microfluidic chip 205 is shown with a single inlet 250 and a plurality of outlets 255, but it will be appreciated that multiple inlets 250 or a single outlet 255 may alternatively be used. Inlet 250 is in fluid communication with a channel array of a plurality of microfluidic channels 260 of different cross-sectional dimensions via a branching network of fluid pathways. Each microfluidic channel 260 in the channel array may be in fluid communication with a corresponding outlet 255.

As well treatment fluid is introduced into inlet 250, it passes to each of the different microfluidic channels 260, and the well treatment fluid partitions into different size classes based on the cross-sectional dimension of the corresponding microfluidic channel 260. For example, particles of different sizes can be prevented from flowing through the different microfluidic channels 260 based on the dimensions of the solids. In this context, particles can refer to both solid particles suspended in the well treatment fluid as well as water droplets in an oil-based invert emulsion. Each size class can comprise particles with specific physical and/or chemical properties. In the case of drilling mud, the specific physical and chemical properties of different size classes can be representative of the drilling mud at different instances in time, with the smaller size classes representing later drilling operations. For example, as drilling progresses, larger particles can be grinded into smaller particles, so the smaller size classes can be representative of future properties of the drilling mud.

The flows of the different size classes through the different microfluidic channels 260 can be determined (e.g., by imaging or conductivity measurements as described above), as a function of time, and optionally temperature or pressure, to obtain flow data, imaging data, conductivity data, and optionally temperature dependent flow, imaging, or conductivity data or pressure dependent flow, imaging, or conductivity data, which can explicitly include the size class information. The flow, imaging, or conductivity data can then be used to characterize the fluid properties for each of the size classes. In the case of drilling mud, this can allow for prediction of drilling mud properties at later stages of a drilling operation, allowing for additives to be prepared in advance to optimize the drilling mud properties at later drilling stages.

Microfluidic chip 205 also includes a plurality of actuatable valves or plugs 265, which can be controlled so as to limit or allow the flow of the well treatment fluid through the microfluidic channels 260, and a plurality of pressure sensing ports 270, which can be coupled to pressure transducers to allow for determination or monitoring of the pressure of the well treatment fluid in the microfluidic chip, such as for use in obtaining pressure dependent imaging data. Optionally, each microfluidic channel 260 can be associated with a corresponding actuatable valve or plug 265 and a corresponding pressure port 270 and/or pressure transducer. In some cases, pressure can be controlled by introducing the well treatment fluid into the inlet 250 at a controlled pressure and by closing various actuatable valves or plugs 265 to limit flow of the well treatment fluid through a particular microfluidic channel 260. In some cases, pressure can be controlled by controlling a backpressure at the various outlets 255. The imaging data obtained by imaging the microfluidic channels 260 can provide for an extent of flow (e.g., position or distance of the well treatment fluid in each microfluidic channel 260) as a function of time, and optionally as a function of pressure and/or temperature by repeating the imaging and flowing process for various pressures and/or temperatures.

Microfluidic chip 205 also includes a plurality of electrical contact points 280, such as in one or more of the microfluidic channels 260. Electrical contact points 280 can be used to monitor electrical conductivity of the well treatment fluid, which can give insights as to the position of the well treatment fluid in the microfluidic channels 260 or to the composition of the well treatment fluid. In some examples, electrical conductivity measurements can be used in place of or in addition to imaging techniques for sensing a position of the well treatment fluid as it flows through the microfluidic channels.

The imaging data and/or electrical conductivity data so obtained can be used to determine fluid properties of the well treatment fluid and fluid properties of the different size classes for the well treatment fluid. Fluid properties may include, but are not limited to, rheological properties, chemical properties, or physical properties. For example, fluid properties may include aspects such as, but not limited to, particle size, particle size distributions, alkalinity, viscosity, opacity, or electrical conductivity.

In some examples, the fluid properties may be characterized by a trained-machine learning model. Training of the machine-learning model may use a set of reference fluids of known fluid properties, which are characterized by passing through a microfluidic chip, such as microfluidic chip 205, to obtain imaging data, and optionally pressure dependent imaging data or temperature dependent imaging data. The training of the machine-learning model may generate a set of parameters and one or more functions configured to transform the obtain imaging data, and optionally pressure dependent imaging data or temperature dependent imaging data into the fluid properties. Once trained using a suitable number of reference fluids of known fluid properties the trained machine-learning model can use flow, imaging, or conductivity data, and optionally pressure dependent flow, imaging, or conductivity data or temperature dependent flow, imaging, or conductivity data, for a well treatment fluid as input along with set of parameters and the one or more functions to generate output comprising the fluid properties for each of the different size classes of the well treatment fluid.

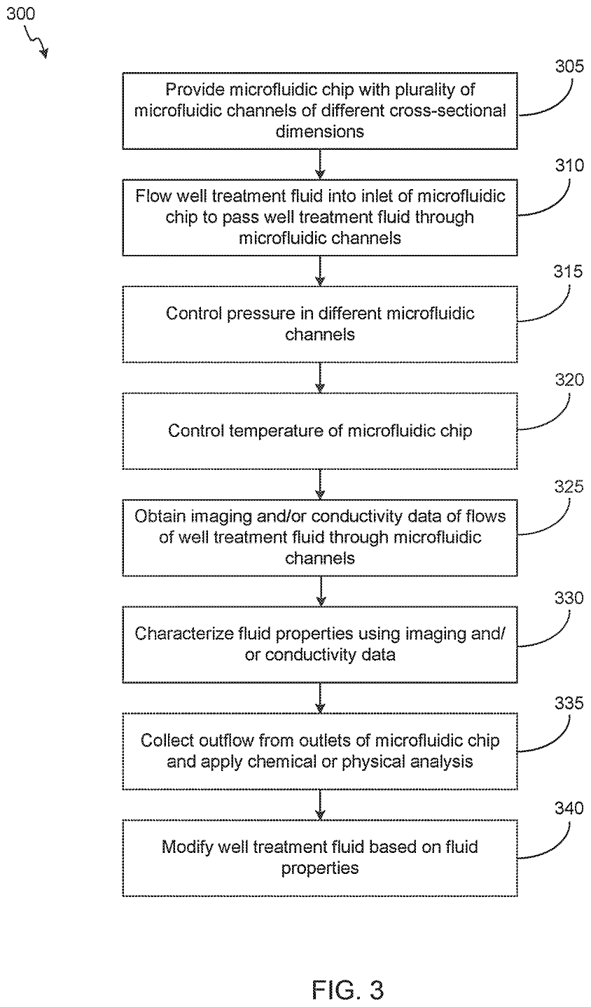

FIG. 3 provides an overview of an example method 300 for characterizing well treatment fluids. Method 300 starts at block 305, where a microfluidic chip is provided, such as a microfluidic chip having a channel array comprising a plurality of microfluidic channels of different cross-sectional dimensions, such as positioned between an inlet and a plurality of corresponding outlets. In a non-limiting example, the microfluidic chip may comprise microfluidic chip 205 described above with reference to FIG. 2. The microfluidic chip may optionally be coupled to one or more pressure transducers, a temperature sensor, a heater or heat pump, and/or one or more pumps, to allow temperatures and pressures of the microfluidic channels to be monitored and/or controlled. The microfluidic chip may be positioned in optical communication with one or more imaging sensors, and optionally one or more light sources, to allow for the microfluidic channels to be imaged. The microfluidic chip may include electrodes or electrical contact points to allow for electrical conductivity of the fluid in the microfluidic channels to be monitored.

At block 310, a well treatment fluid is introduced into an inlet of the microfluidic chip to allow the well treatment fluid to flow to and potentially through the microfluidic channels. The well treatment fluid may be introduced into the inlet using a fluid pump, such as a syringe pump, pressure pump, or other suitable pumping equipment. As the fluid enters the microfluidic chip, the fluid may partition into different size classes based on the cross-sectional dimensions of the various microfluidic channels.

At block 325, imaging data and/or electrical conductivity of flows of the well treatment fluid in the microfluidic channels can be obtained, such as using the imaging device and/or electrodes or electrical contact points. Optionally, at block 315, pressure in the microfluidic channels can be controlled to obtain pressure dependent imaging data. Optionally, at block 320, temperature in the microfluidic channels can be controlled to obtain pressure dependent data. In some cases, the process of flowing the well treatment fluid into the inlet of the microfluidic chip can be repeated one or more times, so as to allow for imaging and/or conductivity data to be collected at different temperatures or pressures. Optionally, the microfluidic chip may contain different microfluidic channels controlled to different pressures or temperatures to allow for obtaining pressure dependent data and/or temperature dependent data in parallel. Optionally, multiple microfluidic chips may be used at the same time to allow for obtaining pressure dependent data and/or temperature dependent data in parallel.

At block 330, the imaging and/or electrical conductivity data is used to characterize the fluid properties of the well treatment fluid or the various different size classes of the well treatment fluid. Example fluid properties include rheological properties, chemical properties, or physical properties, such as particle size, particle size distribution, alkalinity, viscosity, opacity, or electrical conductivity. Characterizing the well treatment fluid can be performed, in some examples, using a trained machine-learning model, which can use parameters learned using reference fluids and associated imaging data, and functions for transforming imaging data into predicted fluid properties using the learned parameters.

Optionally, at block 335, outflow from the outlets of the microfluidic chip may be collected. This outflow, which may correspond to well treatment fluid or size partitioned flows of well treatment fluid can be subjected to additional chemical or physical analysis.

Optionally, at block 340, the well treatment fluid can be augmented, such as by providing one or more additives to the well treatment fluid. The augmentation may be performed based on the fluid properties obtained through the characterization performed at block 330, and optionally using results from any additional chemical or physical analysis. In one example, for the case where the well treatment fluid is drilling mud, additives can be mixed with or into the drilling mud, such as based on the expected future performance by way of the fluid properties obtained for smaller size classes.

Despite having fluid properties for the well treatment fluid, or in the case where fluid properties are not available, it may be desirable to understand how the well treatment fluid may perform. For example, in the case of drilling mud, it may be desirable to understand, in advance of or while using the drilling mud in a well drilling operation, how the drilling mud may perform, such as how well the drilling mud may seal the borehole and an extent to which the drilling mud may generate formation damage. These characteristics may be interpreted, at least in part, based on a thickness of the external mudcake that the drilling mud generates when used in a drilling operation and/or an extent to which the drilling mud invades the rock adjacent to the borehole. In some cases, an internal mudcake can provide information about the extent of formation damage caused by the drilling mud. The present disclosure provides tools for evaluating the performance of well fluids in such a way by using a microfluidic chip that is functionalized as a micromodel for rock in the formation. For example, the microfluidic chip can include pore characteristics, such as pore sizes or pore structures, that are known or expected for rock in the formation.

In some example, photolithography and wet etching can be used to implant a porous domain resembling pore space characteristic of the targeted formation onto regular soda-lime glass substrates. A suitable photomask can then be prepared, followed by spin-coating a positive photoresist on a glass substrate and the ensemble can be soft baked (e.g., put on a hotplate for two minutes at 115° C.). The photomask design can be transferred to the soft baked photoresist using an ultraviolet light system and subsequently hard baked (e.g., heated at 120° C. with a hotplate for two minutes). Hydrofluoric acid can be used to etch the glass according to the implanted porous design. Finally, the etched substrate can be fused to a smooth cheap soda lime glass cover by heating the ensemble gradually up to 700° C. Micromodels can be functionalized by changing their wettability via aging with crude oil or chloro-dimethyloctylsilane coating, incorporating surface roughness, and/or attaching authigenic minerals (e.g., montmorillonite) to the pore surface.

In some examples, a library of microfluidic chips, representing micromodels for different rocks with different characteristics, may be available or included as a part of any of the systems described herein. Such a library of microfluidic chips can be useful for evaluating the well treatment fluid performance prior to or while contacting the rock in the formation with the well treatment fluid, allowing for changes to be made to the well treatment fluid in advance or during a well drilling or completion operation. The library of microfluidic chips can be used to select an appropriate micromodel based on geological interpretations, stratigraphy, core samples from nearby wells, rock cuttings, and/or information obtained when drilling proceeds.

Once sets of micromodels with properties associated with a formation are fabricated or available, well treatment fluid samples can injected into them using a high precision syringe pump or pressure pump, as described above with reference to FIG. 1. In the case of drilling mud, the mud cake deposited at the inlet reservoir and the particulates penetrating the formation can visualized using a stereomicroscope and a digital camera, for example. For high pressure and temperature conditions, the micromodels can be submersed in a sealed flow cell or a backpressure applied to the outlet. In some cases, well treatment fluid performance experiments on multiple micromodels can be conducted simultaneously.

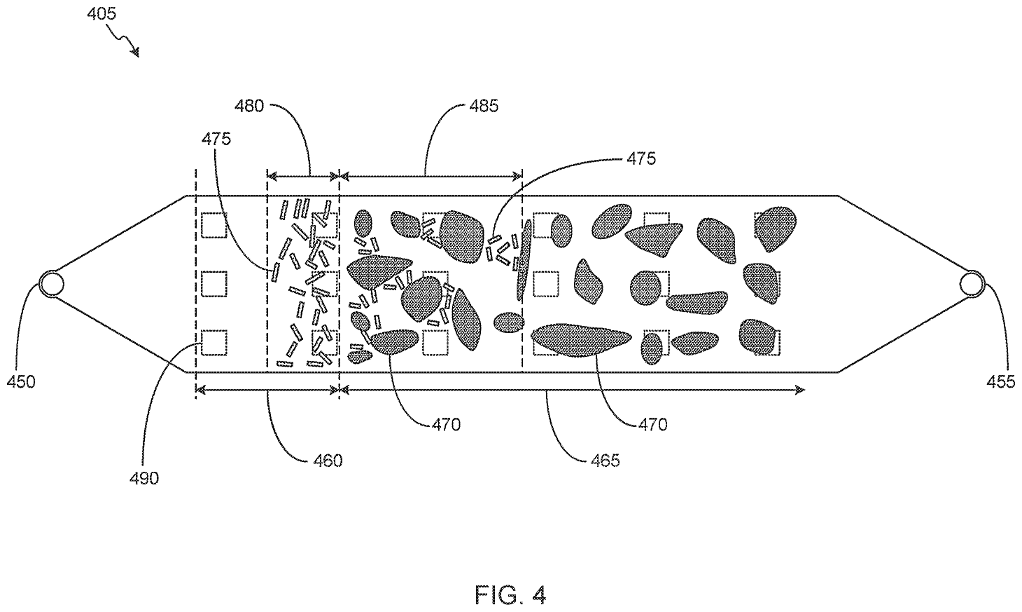

FIG. 4 provides a schematic illustration of an example section of a microfluidic chip 405 functionalized as a model for a wellbore and a formation for evaluating well treatment fluid performance in association with the methods, systems, and techniques described herein. Microfluidic chip 405 is shown with an inlet 450 and an outlet 455, but it will be appreciated that multiple inlets 450 or a multiple outlet 455 may alternatively be used. Inlet 450 is in fluid communication with a microfluidic channel, functionalized in a first region 460 representing a wellbore, such as with little or no obstructions and open space, and in a second region 465 including obstructions 470 representing a formation with appropriate pore characteristics and surface functionality.

Microfluidic chip 405 also includes a plurality of electrical contact points 490, such as at various points along first region 460 and/or second region 465. In some examples, an array of electrical contact points 490 may be included in microfluidic chip 405. Electrical contact points 490 can be used to monitor electrical conductivity of the well treatment fluid, which can give insights as to the position of the well treatment fluid in the microfluidic channels 260 or to the composition of the well treatment fluid. In some examples, electrical conductivity measurements can be used in place of or in addition to imaging techniques for sensing a position of the well treatment fluid as it flows through the microfluidic channels.

As well treatment fluid is introduced into inlet 450, it passes into and through first region 460 and into second region 465. As the well treatment fluid flows into the microfluidic channels, particles 475 may be deposited in the first region 460 and build up, representing, for example, an external mudcake deposited on the walls of a borehole. In some cases, particles 475 may invade the second region 465 and may represent, for example, an internal mudcake deposited in the formation adjacent to the borehole or an invaded zone.

The flows of the well treatment fluid through the microfluidic chip and the build-up of particles 475 in the first region 460 and the second region 465 can be imaged or its electrical conductivity can be measured or monitored, as described above, as a function of time, and optionally temperature or pressure, to obtain flow, imaging, or conductivity data, and optionally temperature dependent flow, imaging, or conductivity data or pressure dependent flow, imaging, or conductivity data. The data can allow for determination of a mudcake thickness 480, H_{mc}, and a diameter of invasion 485, Dᵢ. These characteristics observed in a micromodel can directly represent the expected corresponding characteristics in an actual wellbore and formation or may be scaled to provide expected corresponding characteristics in an actual wellbore and formation.

In some cases the characteristics observed in the micromodel may vary as a function of temperature and/or pressure, and so it may be useful to characterize the expected corresponding characteristics in an actual wellbore and formation using a trained-machine learning model. Training of the machine-learning model may use a set of reference fluids of known performance properties for a particular rock or formation, such as actual mudcake thickness and diameter of invasion. These reference fluids can be characterized by passing through a microfluidic chip, such as microfluidic chip 405, to obtain imaging data and/or electrical conductivity data, and optionally pressure dependent imaging and/or conductivity data or temperature dependent imaging and/or conductivity data. The training of the machine-learning model may generate a set of parameters and one or more functions configured to transform the obtained imaging and/or conductivity data, and optionally pressure dependent data or temperature dependent data into the known characteristics. Once trained using a suitable number of reference fluids of known performance properties, the trained machine-learning model can use imaging and/or conductivity data, and optionally pressure dependent data or temperature dependent data, for a well treatment fluid as input along with set of parameters and the one or more functions to generate output comprising the expected characteristics in an actual wellbore and formation.

With the expected characteristics in hand, this can allow for optimization of the well treatment fluid in real-time or near real-time, allowing for additives to be mixed into the well treatment fluid in advance or during a well drilling or completion operation. In some cases, the additives can be mixed in the well treatment fluid, which can be subjected to testing with the micromodel, as described above, optionally in an iterative fashion, to optimize the well treatment fluid.

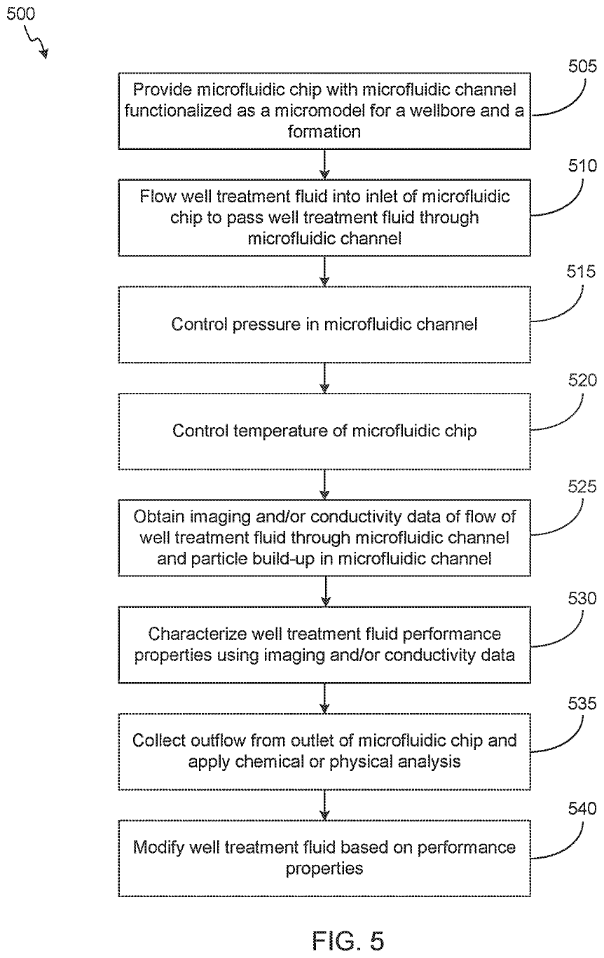

FIG. 5 provides an overview of an example method 500 for characterizing well treatment fluid performance. Method 500 starts at block 505, where a microfluidic chip is provided, such as a microfluidic chip functionalized as a micromodel of a wellbore and a formation. In a non-limiting example, the microfluidic chip may comprise microfluidic chip 405 described above with reference to FIG. 4. The microfluidic chip may optionally be coupled to one or more pressure transducers, a temperature sensor, a heater or heat pump, and/or one or more pumps, to allow temperatures and pressures of the microfluidic channels to be monitored and/or controlled. The microfluidic chip may be positioned in optical communication with one or more imaging sensors, and optionally one or more light sources, to allow for the microfluidic channels to be imaged. The microfluidic chip may be selected from a micromodel library, such as based on geological interpretations, stratigraphy, core samples from nearby wells, rock cuttings, and/or information obtained from a drilling or completion operation to allow the micromodel to match characteristics of a particular formation.

At block 510, a well treatment fluid is introduced into an inlet of the microfluidic chip to allow the well treatment fluid to flow to and potentially through different regions of a microfluidic channel. The well treatment fluid may be introduced into the inlet using a fluid pump, such as a syringe pump, pressure pump, or other suitable pumping equipment. As the fluid enters the microfluidic chip, particles in the fluid may deposit at different regions based on pore characteristics and surface characteristics of the microfluidic chip, as well as the fluid properties, temperature conditions, and/or pressure conditions.

At block 525, imaging data of flows of the well treatment fluid and the build-up of particles in the microfluidic channel can be obtained, such as using the imaging device. Optionally, at block 515, pressure in the microfluidic channel can be controlled to obtain pressure dependent imaging data. Optionally, at block 520, temperature in the microfluidic channel can be controlled to obtain pressure dependent imaging data. In some cases, the process of flowing the well treatment fluid into the inlet of the microfluidic chip can be repeated one or more times, so as to allow for imaging data to be collected at different temperatures or pressures. In some cases, a backflow can be applied to clear deposited particles from the microfluidic chip in preparation for a repeated fluid flow and imaging process. Optionally, the microfluidic chip may contain different microfluidic channels controlled to different pressures or temperatures to allow for obtaining pressure dependent imaging data and/or temperature dependent imaging data in parallel. Optionally, multiple microfluidic chips may be used at the same time to allow for obtaining pressure dependent imaging data and/or temperature dependent imaging data in parallel.

At block 530, the imaging data is used to characterize the performance properties of the well treatment fluid. Characterizing the well treatment fluid performance can be achieved, in some examples, using a trained machine-learning model, which can use parameters learned using reference fluids and associated imaging data, and functions for transforming imaging data into predicted fluid performance properties using the learned parameters.

Optionally, at block 535, outflow from the outlet of the microfluidic chip may be collected. This outflow, which may correspond to filtrate (e.g., mud filtrate in the case of drilling mud as the well treatment fluid) can be subjected to additional chemical or physical analysis.

Optionally, at block 540, the well treatment fluid can be augmented, such as by providing one or more additives to the well treatment fluid. The augmentation may be performed based on the performance properties obtained through the characterization performed at block 530, and optionally using results from any additional chemical or physical analysis of the filtrate. In one example, for the case where the well treatment fluid is drilling mud, additives can be mixed with or into the drilling mud, such as based on the expected thickness of an external mudcake or the expected invasion diameter. The augmented well treatment fluid can optionally be subjected to further characterization, similar to that performed at block 530, to confirm whether the augmentation made the performance properties better or worse, for example.

A computing device may be incorporated as part of the previously described systems, such as systems for characterizing well treatment fluids and for characterizing well treatment fluid performance. Computing devices may be useful for performing aspects of the previously described methods. For example, computing devices may be useful for controlling or measuring pressure, for controlling or measuring temperature, for controlling fluid flow or pumping speed, for obtaining imaging data from an imaging device, and/or for controlling a light source. Computing devices may also be useful for executing machine learning models, such as machine learning models for transforming imaging data into fluid properties or transforming imaging data into performance properties. An example computing device comprises hardware elements that may be electrically coupled via a bus (or may otherwise be in communication). The hardware elements may include one or more processors, including without limitation one or more general-purpose processors and/or one or more special-purpose processors (such as digital signal processing chips, graphics acceleration processors, video decoders, and/or the like); one or more input devices, which may include without limitation a mouse, a touchscreen, keyboard, remote control, voice input, and/or the like; and one or more output devices, which may include without limitation a display device, a printer, speaker, a servo, a linear actuator, a rotational actuator, etc.

The computing device may further include (and/or be in communication with) one or more non-transitory storage devices, which may comprise, without limitation, local and/or network accessible storage, and/or may include, without limitation, a disk drive, a drive array, an optical storage device, a solid-state storage device, such as a solid state drive (“SSD”), random access memory (“RAM”), and/or a read-only memory (“ROM”), which may be programmable, flash-updateable and/or the like. Such storage devices may be configured to implement any appropriate data stores, including without limitation, various file systems, database structures, and/or the like.

The computing device may also include a communications subsystem, which may include without limitation a modem, a network card (wireless or wired), an infrared communication device, a wireless communication device, and/or a chipset (such as a Bluetooth device, a Bluetooth Low Energy or BLE device, an 802.11 device, an 802.15.4 device, a WiFi device, a WiMax device, cellular communication device, etc.), a G.hn device, and/or the like. The communications subsystem may permit data to be exchanged with a network, other computer systems, and/or any other devices described herein. In many embodiments, the computing device will further comprise a working memory, which may include a RAM or ROM device, as described above.

The computing device also may comprise software elements, such as located within the working memory, including an operating system, device drivers, executable libraries, and/or other code, such as one or more application programs, which may comprise computer programs provided by various embodiments, and/or may be designed to implement methods, and/or configure systems, provided by other embodiments, as described herein. Merely by way of example, one or more procedures described with respect to the methods discussed above may be implemented as code and/or instructions executable by a computer (and/or a processor within a computer); in an aspect, then, such code and/or instructions may be used to configure and/or adapt a computer (or other device) to perform one or more operations in accordance with the described methods.

A set of these instructions and/or code may be stored on a non-transitory computer-readable storage medium, such as the non-transitory storage devices described above. In some cases, the storage medium may be incorporated within a computer system, such as the computing device described above. In other embodiments, the storage medium may be separate from a computer system (e.g., a removable medium, such as a compact disc, or a cloud- or network-based storage system), and/or provided in an installation package, such that the storage medium may be used to program, configure, and/or adapt a computer with the instructions/code stored thereon. These instructions may take the form of executable code, which is executable by the computing device or a component thereof and/or may take the form of source and/or installable code, which, upon compilation and/or installation on the computing device (e.g., using any of a variety of generally available compilers, installation programs, compression/decompression utilities, etc.), then takes the form of executable code.

It will be apparent to those skilled in the art that substantial variations may be made in accordance with specific requirements. For example, customized hardware may also be used, and/or particular elements may be implemented in hardware, software (including portable software, such as applets, etc.), or both. Further, connection to other computing devices such as network input/output devices may be employed.

As mentioned above, in one aspect, some embodiments may employ a computing device to perform methods in accordance with various embodiments. According to a set of embodiments, some or all of the procedures of such methods are performed by the computing device in response to a processor executing one or more sequences of one or more instructions (which may be incorporated into the operating system and/or other code, such as an application program) contained in the working memory. Such instructions may be read into the working memory from another computer-readable medium, such as one or more non-transitory storage devices. Merely by way of example, execution of the sequences of instructions contained in the working memory may cause the processor to perform one or more procedures of the methods described herein.

The terms “machine-readable medium,” “computer-readable storage medium” and “computer-readable medium,” as used herein, refer to any medium that participates in providing data that causes a machine to operate in a specific fashion. These mediums may be non-transitory. In an embodiment implemented using the computing device, various computer-readable media may be involved in providing instructions/code to a processor for execution and/or may be used to store and/or carry such instructions/code. In many implementations, a computer-readable medium is a physical and/or tangible storage medium. Such a medium may take the form of a non-volatile media or volatile media. Non-volatile media include, for example, optical and/or magnetic disks, such as a non-transitory storage device. Volatile media include, without limitation, dynamic memory, such as the working memory.

Common forms of physical and/or tangible computer-readable media include, for example, a floppy disk, a flexible disk, hard disk, magnetic tape, or any other magnetic medium, a CD-ROM, any other optical medium, any other physical medium with patterns of marks, a RAM, a PROM, EPROM, a FLASH-EPROM, any other memory chip or cartridge, or any other medium from which a computer may read instructions and/or code. Network-based and cloud-based storage systems may also be useful forms of computer-readable media.

Various forms of computer-readable media may be involved in carrying one or more sequences of one or more instructions to the processor for execution. Merely by way of example, the instructions may initially be carried on a magnetic disk and/or optical disc of a remote computer. A remote computer may load the instructions into its dynamic memory and send the instructions as signals over a transmission medium to be received and/or executed by the computing device.

The communications subsystem (and/or components thereof) generally will receive signals, and the bus then may carry the signals (and/or the data, instructions, etc. carried by the signals) to the working memory, from which the processor retrieves and executes the instructions. The instructions received by the working memory may optionally be stored on a non-transitory storage device either before or after execution by the processor.

It should further be understood that the components of computing device may be distributed. For example, some processing may be performed in one location using a first processor while other processing may be performed by another processor remote from the first processor. Optionally, systems described herein may include multiple independent processors that may exchange instructions or issue commands or provide data to one another. Other components of computing device may be similarly distributed. As such, a computing device may be interpreted as a distributed computing system that performs processing in multiple locations. In some instances, a computing device may be interpreted as a single computing device, such as a distinct laptop, desktop computer, or the like, depending on the context.

### REFERENCES

U.S. Pat. Nos. 5,253,719, 9,898,559, 9,951,613.

U.S. Patent Application Publication Nos. US 2019/0219728, US 2005/0037508.

Mehmani et al., 2019, “Capillary Trapping Following Imbibition in Porous Media: Microfluidic Quantification of the Impact of Pore-Scale Surface Roughness,” Water Resources Research, Vol. 55, 9905-9925. DOI: 10.1029/2019WR025170

Mehmani et al., 2019, “Residual oil saturation following gas injection in sandstones: Microfluidic quantification of the impact of pore-scale surface roughness,” Fuel, 251, 147-161. DOI: 10.1016/j.fuel.2019.02.118

Du et al., 2019, “Micromodel Study of the Impacts of Fracture Connectivity and Wettability on Matrix Sweep Efficiency,” Paper presented at the SPE/AAPG/SEG Unconventional Resources Technology Conference, Denver, Colorado, USA 22-24 Jul. 2019 DOI: 10.15530/urtec-2019-144

Caenn et al., 2017, “Chapter 3, Evaluating Drilling Fluid Performance” in “Composition and properties of drilling and completion fluids,” Gulf Professional Publishing. DOI: 10.1016/B978-0-12-804751-4.00003-1

Riordon et al., 2019, “Deep Learning with Microfluidics for Biotechnology,” Trends in Biotechnology, 37(3), 310-324. DOI: 10.1016/j.tibtech.2018.08.005

Yuan et al., 2018 “A comprehensive review of formation damage during enhanced oil recovery,” Journal of Petroleum Science and Engineering, 167, 287-299. DOI: 10.1016/j.petrol.2018.04.018

Porter et al., 2015, “Geo-material microfluidics at reservoir conditions for subsurface energy resource applications,” Lab on a Chip 15(20), 4044-4053. DOI: 10.1039/C5LC00704F

Mejia et al., 2019, “Surfactant flooding in oil-wet micromodels with high permeability fractures,” Fuel, 241, 1117-1128. DOI: 10.1016/j.fuel.2018.12.076

Schroeder et al., 2019, “Experimental Method for Time-Lapse Micro-CT Imaging of Mud-Filtrate Invasion and Mudcake Deposition,” Petrophysics, 60, 620-630. DOI: 10.30632/PJV60N5-2019a6

Bourgoyne Jr, A. T., Millheim, K. K., Chenevert, M. E. and Young Jr, F. S., 1986. Applied drilling engineering. Volume 2. ISBN 978-1-5563-001-0

Song et al., 2016, “Direct visualization of pore-scale fines migration and formation damage during low-salinity waterflooding,” Journal of Natural Gas Science and Engineering, 34, 1276-1283. DOI: 10.1016/j.jngse.2016.07.055

Yun et al., 2020, “Toward Reservoir-on-a-Chip: Rapid Performance Evaluation of Enhanced Oil Recovery Surfactants for Carbonate Reservoirs Using a Calcite-Coated Micromodel,” Scientific Reports, 10, 782. DOI: 10.1038/s41598-020-54785-x

Fiorini et al., 2018, “Disposable microfluidic devices: fabrication, function, and application,” Biotechniques, 38, 429-446. DOI: 10.2144/05383RV02

### STATEMENTS REGARDING INCORPORATION BY REFERENCE AND VARIATIONS

All references throughout this application, for example patent documents including issued or granted patents or equivalents, patent application publications, and non-patent literature documents or other source material are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “1, 2, 3, 1 and 2, 1 and 3, 2 and 3, or 1, 2, and 3”.

Every formulation or combination of components described or exemplified can be used to practice the invention, unless otherwise stated. Specific names of materials are intended to be exemplary, as it is known that one of ordinary skill in the art can name the same material differently. It will be appreciate that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method for characterizing well treatment fluids, the method comprising:
providing a microfluidic chip, the microfluidic chip comprising:
at least one inlet and a plurality of outlets, and
a channel array comprising plurality of microfluidic channels having different cross-sectional dimensions, wherein the at least one inlet is in fluid communication with the plurality of microfluidic channels, and wherein the plurality of microfluidic channels are in respective fluid communication with the plurality of outlets;
flowing a well treatment fluid into the at least one inlet to pass the well treatment fluid to the plurality of microfluidic channels, wherein the well treatment fluid partitions into a plurality of different size classes based on a cross-sectional dimension of a respective microfluidic channel;
obtaining imaging data of flows of the well treatment fluid in the plurality of microfluidic channels; and
characterizing fluid properties for the well treatment fluid or for each of the different size classes using the imaging data.

2. The method of claim 1, further comprising controlling a temperature of the plurality of microfluidic channels to obtain temperature dependent imaging data of flows of the well treatment fluid in the plurality of microfluidic channels, wherein characterizing the fluid properties comprises using the temperature dependent imaging data.

3. The method of claim 1, further comprising monitoring pressures at the plurality of microfluidic channels while obtaining the imaging data, wherein characterizing the fluid properties comprises using the imaging data and the pressures.

4. The method of claim 1, further comprising controlling a backpressure at the plurality of outlets to obtain pressure dependent imaging data of flows of the well treatment fluid in the plurality of microfluidic channels, wherein characterizing the fluid properties comprises using the pressure dependent imaging data.

5. The method of claim 1, wherein obtaining the imaging data of flows of the well treatment fluid in the plurality of microfluidic channels comprises imaging positions of the well treatment fluid in the plurality of microfluidic channels as a function of time.

6. The method of claim 1, wherein characterizing the fluid properties comprises applying the imaging data as input to a trained machine-learning model for determining well treatment fluid properties.

7. The method of claim 6, wherein the trained machine-learning model comprises:
a set of parameters that were learned using a set of reference fluids, each reference fluid of the set of reference fluid corresponding to a previously characterized well treatment fluid or well treatment fluid component, and parameters of the set of parameters describing fluid properties of one or more reference fluids; and
one or more functions configured to transform the input into predicted fluid properties using the set of parameters.

8. The method of claim 6, wherein the trained machine-learning model generates outputs comprising the fluid properties for the well treatment fluid or for each of the different size classes, or wherein the input comprises one or more of:
pressures at the plurality of microfluidic channels,
temperature dependent imaging data, or
pressure dependent imaging data.

9. (canceled)

10. The method of claim 1, wherein the well treatment fluid comprises drilling mud, a cleaning fluid, a casing fluid, or a reservoir fluid, wherein the fluid properties comprises one or more rheological properties, one or more chemical properties, or one or more physical properties, or wherein the fluid properties comprises one or more of a particle size, a size distribution of particles, an alkalinity, a viscosity, an opacity, or an electrical conductivity.

11. (canceled)

12. (canceled)

13. The method of claim 1, further comprising collecting outflow from one or more of the plurality of outlets and applying a chemical analysis or physical analysis to the outflow.

14. A system for characterizing well treatment fluids, the system comprising:
a microfluidic chip, the microfluidic chip comprising:
at least one inlet and a plurality of outlets, and
a channel array comprising plurality of microfluidic channels having different cross-sectional dimensions, wherein the at least one inlet is in fluid communication with the plurality of microfluidic channels, and wherein the plurality of microfluidic channels are in respective fluid communication with the plurality of outlets;
a pump in fluid communication with the at least one inlet for passing the well treatment fluid to the plurality of microfluidic channels to partition the well treatment fluid into a plurality of different size classes based on a cross-sectional dimension of a respective microfluidic channel;
an imaging sensor in optical communication with the microfluidic chip for obtaining imaging data of flows of the well treatment fluid in the plurality of microfluidic channels; and
a computing system for analyzing the imaging data to characterize fluid properties for the well treatment fluid or for each of the different size classes.

15. The system of claim 14, further comprising one or more of:
a pressure transducer coupled to the microfluidic chip for determining pressures in the plurality of microfluidic channels;
a heater in thermal communication with the microfluidic chip for controlling a temperature in the plurality of microfluidic channels; or
a light source in optical communication with the microfluidic chip for illuminating the plurality of microfluidic channels.

16. The system of claim 14, wherein the computing system comprises a processor in data communication with the imaging sensor, and a non-transitory computer readable storage medium in data communication with the processor, the non-transitory computer readable storage medium comprising instructions that, when executed by the processor, cause the processor to perform operations including:
obtaining imaging data of flows of the well treatment fluid in the plurality of microfluidic channels using the imaging sensor; and
characterizing fluid properties for the well treatment fluid or for each of the different size classes using the imaging data.

17. The system of claim 16, wherein the operations further include controlling a temperature of the plurality of microfluidic channels using a heater to obtain temperature dependent imaging data of flows of the well treatment fluid in the plurality of microfluidic channels, wherein characterizing the fluid properties comprises using the temperature dependent imaging data, or wherein the operations further include monitoring pressures at the plurality of microfluidic channels using a pressure transducer while obtaining the imaging data, wherein characterizing the fluid properties comprises using the imaging data and the pressures.

18. (canceled)

19. The system of claim 16, wherein characterizing the fluid properties comprises applying the imaging data as input to a trained machine-learning model for determining well treatment fluid properties.

20. The system of claim 19, wherein the trained machine-learning model comprises:
a set of parameters that were learned using a set of reference fluids, each reference fluid of the set of reference fluid corresponding to a previously characterized well treatment fluid or well treatment fluid component, and parameters of the set of parameters describing fluid properties of one or more reference fluids; and
one or more functions configured to transform the input into predicted fluid properties using the set of parameters.

21. The system of claim 19, wherein the trained machine-learning model generates outputs comprising the fluid properties for the well treatment fluid or for each of the different size classes, or wherein the input comprises one or more of:
pressures at the plurality of microfluidic channels,
temperature dependent imaging data, or
pressure dependent imaging data.

22. (canceled)

23. The system of claim 14, wherein the well treatment fluid comprises drilling mud, a cleaning fluid, a casing fluid, or a reservoir fluid, wherein the fluid properties comprises one or more rheological properties, one or more chemical properties, or one or more physical properties, or wherein the fluid properties comprises one or more of a particle size, a size distribution of particles, an alkalinity, a viscosity, or an opacity.

24. (canceled)

25. (canceled)

26. A method for characterizing performance of well treatment fluids, the method comprising:
providing a microfluidic chip, the microfluidic chip comprising:
an inlet and an outlet, and
a microfluidic channel, wherein the microfluidic channel is in fluid communication with and between the inlet and the outlet, and wherein the microfluidic channel is functionalized as a model for a wellbore and a reservoir;
flowing a well treatment fluid into the inlet to pass the well treatment fluid to the microfluidic channel;
obtaining imaging data of flow of the well treatment fluid in the microfluidic channel; and
characterizing performance properties of the well treatment fluid in the microfluidic channel using the imaging data.

27.-37. (canceled)

38. A system for characterizing performance of well treatment fluids, the system comprising:
a microfluidic chip, the microfluidic chip comprising:
an inlet and an outlet, and
a microfluidic channel in fluid communication with and between the inlet and the outlet, wherein the microfluidic channel is functionalized as a model for a wellbore and a reservoir;
a pump in fluid communication with the inlet for flowing a well treatment fluid to the microfluidic channel;
an imaging sensor in optical communication with the microfluidic chip for obtaining imaging data of flow of the well treatment fluid in the microfluidic channel; and
a computing system for analyzing the imaging data to characterize performance properties of the well treatment fluid in the microfluidic channel.

39.-48. (canceled)
